(19) United States
(12) Patent Application Publication
(10) Pub. No.: US 2008/0249400 A1
(43) Pub. Date: Oct. 9, 2008

(54) Intraoperative Imaging Of Hepatobiliary Structures

(75) Inventors: Dragan Golijanin, Rochester, NY (US); Ronald Wood, Rochester, NY (US); Jay E. Reeder, East Rochester, NY (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: University of Rochester Medical Center, Rochester, NY (US)

(21) Appl. No.: 11/868,432

(22) Filed: Oct. 5, 2007

Related U.S. Application Data
(60) Provisional application No. 60/850,041, filed on Oct. 6, 2006.

(51) Int. Cl. A61B 5/00

(52) U.S. Cl. 600/431

(57) ABSTRACT

The invention provides methods for visualizing hepatobiliary structures, or lesions in a liver, intraoperatively by use of fluorescent dyes.

### CROSS-REFERENCES TO RELATED APPLICATIONS

This application claims the benefit of U.S. Provisional Application No. 60/850,041, filed Oct. 6, 2006, the contents of which are incorporated herein by reference.

### STATEMENT AS TO RIGHTS TO INVENTIONS MADE UNDER FEDERALLY SPONSORED RESEARCH AND DEVELOPMENT

Not applicable.

### REFERENCE TO A “SEQUENCE LISTING,” A TABLE, OR A COMPUTER PROGRAM LISTING APPENDIX SUBMITTED ON A COMPACT DISK.

Not applicable

### BACKGROUND OF THE INVENTION

Laparoscopic cholecystectomy (removal of the gall bladder) represents the most common gastrointestinal surgical procedure, with some 700,000 operations annually performed in the United States. The laparoscopic approach has become the treatment of choice for symptomatic cholelithiasis because of its advantages compared to the classic open technique, in terms of shorter hospital stay, reduced postoperative pain and quicker return to normal daily activities (Deziel et al., Am J Surg, 165:9-14 (1993); Hannan et al., Surgery, 125:223-231 (1999)). The advent of laparoscopic cholecystectomy has resulted in a renewed concern about bile duct injury. Population-based studies have shown in fact a significant increase in the incidence of injury from 2 to 4.5 times higher with the laparoscopic approach when compared to the traditional open cholecystectomy (Fletcher et al., Ann Surg, 229:449-457 (1999); Cohen et al., CMAJ, 154:491-500 (1996); Russell et al., Arch Surg, 131:382-388 (1996); Strasberg et al., J Am Coll Surg, 180:101-125 (1995)).

Unfortunately, common complications of cholecystectomy include injury to the common bile duct, the common hepatic duct, or to the right hepatic duct. Bile duct injury, for example, is associated with significant perioperative morbidity, and mortality (Savader et al., Ann Surg, 225:268-273 (1997); Moossa et al., Arch Surg, 125:1028-1031 (1990)) and high rates of subsequent litigation (Kern K. A, Arch Surg, 132:392-398 (1997)). In the United States, 34.1% of 1661 surgeons responding to a survey reported at least one major biliary injury during laparoscopic cholecystectomy (Archer et al., Ann Surg, 234:549-559 (2001)) and there are data demonstrating that the injury rate remains relatively constant at a high incidence (Adamsen et al., J Am Coll Surg., 184:571-8 (1997); Wudel et al., Am Surg., 67:557-64 (2001)) between newly trained and experienced surgeons, with a frequency of 1 in 200 to 300 operations.

The commonest cause of common bile duct injury is misidentification of the biliary anatomy (70-80% of injuries) (Hugh T. B., Surgery, 132:826-835 (2002)). Seventy percent of surgeons regard bile duct injury as an unavoidable complication and there is evidence to suggest that in the majority of duct injuries the surgeon was erroneously convinced he or she correctly identified the damaged duct. Several techniques have been described to prevent injury (Hunter J. G., Am J Surg, 162:71-76 (1991); Troidl H., World J Surg, 23:846-855 (1999)). The main requirement to apply these techniques is the correct interpretation of the biliary structures.

Currently, once the gall bladder is removed during laparoscopic or open procedures, the transected cystic duct is cannulated for the intraoperative administration of radio opaque contrast material, a procedure called cholangiography. A radiological (x-ray) study is then taken to determine whether the common bile duct is patent. This is a time-consuming process in the operating room, is operator-dependent (requiring a radiologist, a technician, or both), and can produce complications. In most patients, no obstruction is found. Thus, in many cases, the procedure raises costs and risk to the patient without providing a significant additional benefit. Similarly, in cases where the common bile duct is obstructed, endoscopic stone removal is guided with radiologic methods with the same attendant concerns. Furthermore, intraoperative assessment is sometimes complicated, needs repeated x-ray imaging and often carries variability in interpretation between observers.

The routine use of intraoperative cholangiography performed during laparoscopic surgery has been shown to decrease the risk of injury on three population-based studies (Fletcher et al., Ann Surg, 229:449-457 (1999); Flum et al., JAMA, 289:1639-1644 (2003); Flum et al., Arch Surg 136:1287-1292 (2001)). Fletcher et al. report an eightfold lower risk of injury in the presence of complicated gallstone disease (Fletcher et al., supra), while Flum et al. showed a twofold reduction in bile duct injury with the routine use of cholangiography during the learning curve (Flum et al., 2001, supra). Other authors have rejected the systematic use of cholangiography as very low rates of duct injury have been demonstrated in large series of laparoscopic cholecystectomies in which operative cholangiography was rarely used (Barkun, J. S. et al., Ann Surg 218:371-379 (1993); Taylor, O. M. et al., Ann R Coll Surg Engl. 79:376-80 (1997); Wright, K. D. et al., Br J Surg. 85:191-4 (1998)). Strasberg et al., in a review of the literature also concluded that there are no substantial data to support the belief that operative cholangiography prevents bile duct injury (Strasberg et al., J Am Coll Surg, 180:101-125 (1995)). Furthermore, operative cholangiography increases the operative time and is related to a small risk of complications (Voyles, C. R. et al., Ann Surg. 219:744-52 (1994)).

Despite the debate about routine intraoperative cholangiography for prevention of injury, the data suggest that early recognition of injury directly affects patient outcome and that cholangiography may have a role in early recognition of unsuspected duct injury. The data suggest that as much as 90% of duct injuries are missed during surgery (Abdel, Wahab M., Hepatogastroenterology 43:1141-1147 (1996)). In most of these cases, the surgeon is erroneously satisfied that the biliary structures have been correctly identified and has difficulty interpreting the significance of subsequent events like biliary leakage or jaundice, leading to substantial delay in the diagnosis.

Tumors of the biliary tract are uncommon but serious problems. Bile duct cancer is more common in Israel, Japan, and in American Indians than in the general US population. The prevalence of carcinoma of the gall bladder and bile ducts in England and Wales is 2.8 cases per 100,000 females and 2 cases per 100,000 males. The spectrum of lesions ranges from benign tumors, such as adenomas, to malignant lesions, such as adenocarcinomas. Cholangiography via a transhepatic or endoscopic approach is required to define the biliary anatomy and extent of the lesion. Magnetic resonance (MR) or magnetic resonance cholangiography (MRCP) is a noninvasive alternative available in an increasing number of centers. Cholangiography is indicated in any patient who is cholestatic with nondilated bile ducts when the diagnosis is in doubt. The choice of cholangiographic investigation depends on the site of the tumor. In proximal lesions, percutaneous transhepatic cholangiography defines the extent of the tumor and allows for the preoperative placement of percutaneous catheters. Endoscopic retrograde cholangiopancreatography (ERCP) is of greater value in the diagnosis of distal tumors and permits the placement of endoprostheses. Complicated pre and intraoperative imaging of Klatksin tumors, cholangiocarcinomas, is not standardized and is complicated with postprocedural infections or tumor seeding if percutaneous cholangiography is performed, or is inadequate at best when MR or MRCP is used. Pre- and intra-operative imaging of cholangiocarcinomas is crucial for operative planning of liver resection and hepatic duct stenting if the case is deemed unresectable.

A variety of medical techniques suitable for imaging biological tissues and organs are known. These include traditional x-rays, ultra-sound, magnetic resonance imaging (MRI), and computerized tomography (CT). Techniques such as MRI, micro-CT, micro-positron emission tomography (PET), and single photon emission computed tomography (SPECT) have been explored for imaging function and processes in small animals or in vivo, intra operatively. These technologies offer deep tissue penetration and high spatial resolution, but are costly and time consuming to implement.

It would be desirable to have less cumbersome techniques for visualization of hepatobiliary structures which can facilitate the correct identification of the bile duct anatomy. The present invention fills these and other needs.

### BRIEF SUMMARY OF THE INVENTION

In a first group of embodiments, the invention provides methods of visualizing a hepatobiliary structure in a subject during a surgical operation. The methods comprise, within eight hours prior to, or during, the operation, administering by subcutaneous injection, intramuscular injection, or slow, continuous infusion, a dye which fluoresces at an emission wavelength when said dye is contacted with an excitation wavelength; exposing the hepatobiliary structure during the operation to a source of illumination comprising the excitation wavelength such that the fluorescent dye fluoresces; and detecting the fluorescence of the dye, thereby visualizing the hepatobiliary structure during the surgical operation. In some embodiments, the hepatobiliary structure is the bile duct, the common hepatic duct or the right hepatic duct. In some embodiments, the hepatobiliary structure is the gall bladder or bile duct and the surgical operation is a cholecystectomy. In some embodiments, the gall bladder or bile duct, or both, are inflamed. In some embodiments, the hepatobiliary structure is the liver and the surgical operation is a resection of the liver for a living-related liver transplantation. In some embodiments, the hepatobiliary structure is the liver and the surgical operation is transplantation of a donor liver. In some embodiments, the hepatobiliary structure is the common bile duct and the surgical operation is removal of a stone from said bile duct. In some embodiments, the surgical operation is performed using a laparoscopic instrument. In some embodiments, the dye is administered one hour or less prior to said surgical operation. In some embodiments, the dye is administered at the time of anesthesia induction. In some embodiments, the dye is a near infrared dye. In some embodiments, the indocyanine green.

In a second group of embodiments, the invention provides methods of determining, during a surgical operation to remove a patient's gall bladder, whether the patient's common bile duct is open following removal of the gall bladder, comprising: after the gall bladder has been removed, but during the surgical operation, administering systemically a bolus of a dye which fluoresces at an emission wavelength when said dye is contacted with an excitation wavelength; exposing the common bile duct to a source of illumination comprising the excitation wavelength such that the fluorescent dye fluoresces; and detecting the presence or absence of fluorescence of the dye throughout said common bile duct, wherein presence of fluorescence of the dye throughout said common bile duct indicates that the common bile duct is open and wherein absence of fluorescence of the dye throughout the common bile duct indicates that the common bile duct is not open. In some embodiments, the surgical operation is performed using a laparoscopic instrument. In some embodiments, the dye is a near infrared dye. In some embodiments, the near infrared dye is indocyanine green.

In a further group of embodiments, the invention provides methods of assessing, during transplantation of a liver into an abdomen of a subject, blood flow in said transplanted liver, comprising: after said transplanted liver has had blood vessels of the subject connected to it, administering systemically before closing said abdomen a bolus of a dye which fluoresces at an emission wavelength when the dye is contacted with an excitation wavelength; exposing the liver to a source of illumination comprising the excitation wavelength such that the fluorescent dye fluoresces; and detecting the presence or absence of fluorescence of the dye throughout the liver, wherein presence of fluorescence of the dye throughout the liver indicates that blood flow throughout the liver is satisfactory and wherein absence of fluorescence of the dye in some or all of said liver indicates that the blood flow is not satisfactory. In some embodiments, the dye is a near infrared dye. In some embodiments, the near infrared dye is indocyanine green.

In yet a further group of embodiments, the invention provides methods of detecting the presence or absence of a lesion or tumor in a liver in a subject, comprising: administering to said subject a bolus of a dye which fluoresces at an emission wavelength when said dye is contacted with an excitation wavelength; providing an interval of time to permit said dye to partially wash out of said liver; exposing said liver to a source of illumination comprising said excitation wavelength such that any fluorescent dye in said liver fluoresces; detecting the presence or absence of fluorescence of said dye throughout said liver, wherein presence of fluorescence of said dye evenly throughout said liver indicates the absence of an injury or tumor and differences in fluorescence between areas of said liver denote the presence of a lesion or tumor in said liver. In some embodiments, the presence of a lesion is shown by an area of the liver fluorescesing at a level different from that of the majority of the liver. In some embodiments, one area of the liver is flouresan absence of fluorescence in a first area of said liver, which first area is bounded by a second area of said liver that is fluorescing, indicates the presence of a lesion in said first area. In some embodiments, this is evidenced by an abundance of fluorescence in a first area of said liver, which first area is bordered by a second, larger area comprising the majority of said liver that is fluorescing at a lower level than that of said first area, thereby indicating the presence of a lesion in said first area. In some embodiments, the dye is a near infrared dye. In some embodiments, the near infrared dye is indocyanine green.

### BRIEF DESCRIPTION OF THE DRAWINGS

FIG. 1. FIG. 1 is a composite of photographs of the liver of an adult male pig. Portions of the liver had been subjected to thermal ablation by an RF needle electrode. The lighter areas to the right and bottom sides of the photos indicate normal liver tissue. The darker area in the middle show low perfusion areas, while the black area on the left side of the photos had no visible perfusion and was likely dead. Upon palpation, the light areas were flexible and normal, while the darker areas and the black area were relatively stiff.

FIG. 2. FIG. 2 is a photograph of the liver of a male Sprague-Dawley rat to which ICG was administered. The liver was illuminated by laser excitation light delivered by fiber optics through a laparascope. The brightly fluorescing structure is the liver; the bowel is to the right.

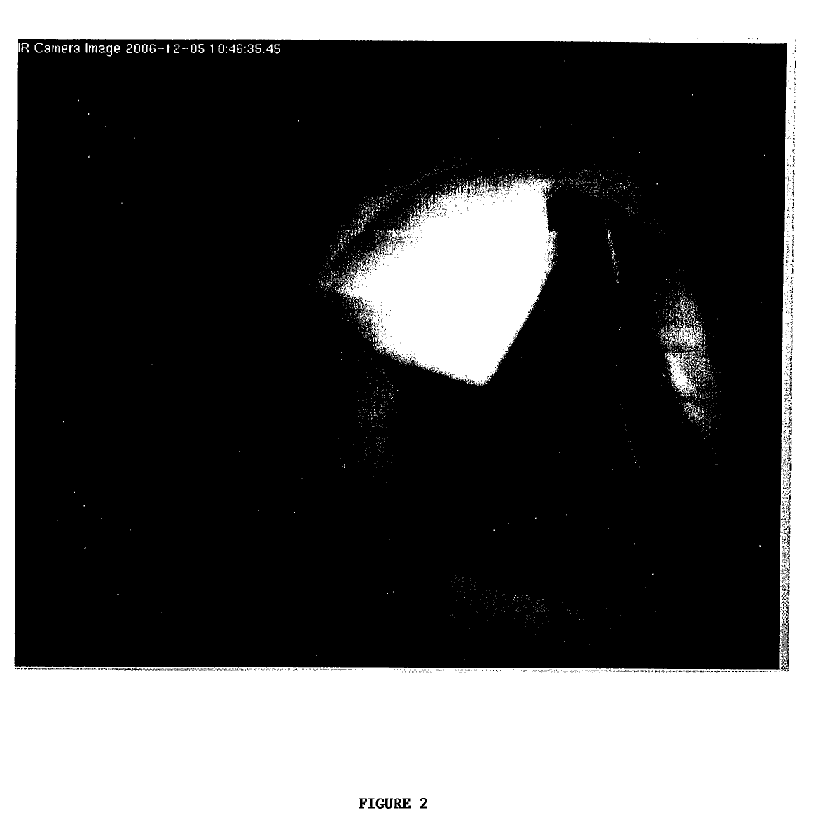

### DETAILED DESCRIPTION OF THE INVENTION

As noted in the Background, laparoscopic surgery for removal of gall stones and clearance of biliary obstruction is the most common gastrointestinal surgical procedure. Typically, once the gall bladder is removed during laparoscopic or open procedures (cholecystectomy), the transected cystic duct is cannulated with a catheter for the intraoperative administration of radio opaque contrast material (cholangiography). A radiological (x-ray) study is then taken to determine whether the duct is patent. This is a time-consuming process in the operating room, is operator-dependent (requiring a radiologist and technician), and may result in complications. In most patients no obstruction is found therefore it inflicts cost, expense and may result in additional risk without significant additional benefit. Similarly, in cases where the common bile duct is obstructed, endoscopic stone removal is guided with radiologic methods with the same attendant concerns.

In studies using the fluorescent dye indocyanine green (“ICG”) to image nerves in the penis, we observed that the liver also fluoresced. We realized that this fluorescence could be used to solve several surgical problems associated with the liver and associated anatomical structures. The removal of the fluorescent dye indocyanine green (ICG) is known, however, to be related to liver-dependent circulation and excretion via the biliary tree to the bowel. Upon introduction of ICG, the vasculature is promptly defined, followed shortly by fluorescence of the liver and the appearance of fluorescence in bile.

Unfortunately, the liver is densely vascularized, and intravenous or other systemic administration of ICG before the surgical procedure results in such intense fluorescence of the liver vasculature that it may be difficult to see the biliary ducts and other anatomic structures around the liver against the background. This problem is further aggravated by the fact that the conditions necessitating such surgeries often involve inflammation of the gall bladder, which makes it even harder to tell the structures apart even under normal illumination and makes it even easier to cut the wrong tissues, such as the common bile duct, the common hepatic duct, and the right hepatic duct. Further, systemically administered dye is quickly cleared from the circulation, which can make it difficult to visualize smaller structures while maintaining dye concentrations that do not produce intense background fluorescence. Thus, even if systemic administration of a fluorescent dye, such as ICG, is normally suitable for the visualization of organs during surgery, it is not suitable prior to cutting one or more of these ducts for visualizing the duct or ducts and helping guide the surgical team to improve the outcome of such surgeries.

The methods of the present invention solve the problems unsolved by prior techniques of visualizing organ structures with fluorescent dyes. The methods change the route and timing of administration so that fluorescent dyes can be used to observe biliary obstructions and anatomical anomalies. In some embodiments, to visualize the common biliary duct, the common hepatic duct, the right hepatic duct, or to see a stone in the common biliary duct, the dye is administered subcutaneously (sometimes abbreviated as “SubQ,” “SQ,” or “SC”)). This limits the rate of dye introduction and prevents the fluorescence of the liver vasculature and parenchyma from becoming so intense that it compromises visualization of the ducts or the ability to visualize a stone against the fluorescence of a duct. For open field surgery, the dye is preferably administered up to an hour before visualization of the ducts or other structure is desired and more preferably administered 10 to 15 minutes before. Conveniently, the dye is administered at or around the time of anesthesia induction. For laparoscopic procedures, the dye is preferably administered as soon as the trocars are inserted.

Subcutaneous administration and, in some embodiments, intramuscular administration, is also useful for use in preparations to resect the donor's liver during living-related liver transplantation. Subcutaneous administration of the dye permits localization and evaluation of the liver vasculature and biliary tree, as well as facilitate resection. Further, subcutaneous administration of the dye in the recipient permits improved evaluation of liver perfusion and biliary drainage in the recipient and can facilitate exact reconstruction and reanastomosis and reconstruction of the bile duct. For example, if the liver fluoresces, it means the blood supply to the organ has been properly connected. If only a portion of the liver shows fluorescence, then there is a problem which needs to be rectified.

For visualizing liver tumors and lesions, the dye can likewise be administered subcutaneously to permit identification of malformations and lesions, such as hemangiomas. Small metastatic lesions can be seen easily during laparoscopic illumination. This visualization can be used, for example, to guide a subsequent radical excision, if feasible. During the second and third (cellular and excretory) phases of ICG distribution, tumor visualization is expected to be dramatically enhanced, permitting the surgeon to accurately plan segmental resection resulting in negative margins of resection, as well as removal of satellite lesions.

In another group of embodiments, the problems noted above are addressed by a slow continuous intravenous infusion, rather than the bolus injection usually used for administration of ICG. Conveniently, this is done by a pump, whose rate of infusion can be adjusted and controlled with precision to achieve the desired degree of fluorescence. Such pumps are already in use for administration of other agents during surgery. Typically, the dye is administered at a relatively slow rate and the resulting fluorescence is visualized to determine if illumination of the structure of interest is sufficient without being overwhelmed by fluorescence of surrounding structures.

In some embodiments, once the gall bladder is removed and the cystic duct clipped, a bolus of fluorescent dye, such as ICG, can be administered systemically and the common bile duct and duodenum observed. If fluorescence is observed in the common bile duct, and if fluorescence is observed in the duodenum, then the duct is highly likely to be open (patent) or to have an obstruction that would be considered to be insignificant. (For convenience of reference, patients exhibiting this pattern of fluorescence will be referred to as being in “group 1”.) If the structures do not fluoresce, than the duct is obstructed. (For convenience of reference, patients exhibiting this pattern of fluorescence will be referred to as being in “group 2”.) In cases in which dye drainage through the bile ducts to the duodenum is prolonged, as seen by fluorescence, there is a need for an intraoperative cholangiogram to explore the common bile duct. (For convenience of reference, patients exhibiting this pattern of fluorescence will be referred to as being in “group 3”.) The methods of the invention therefore permit the surgeon to distinguish patients in group 1 from those of groups 2 or 3, and thereby avoid using radiologic procedures, with their attendant risks and costs, on patients in that group.

The use of fluorescent dyes permits separation of blood flow from organ accumulation and excretion. With no dye “on board,” a small injection makes the vessels “illuminate”; the dye then traverses the parenchyma to the gall bladder and ducts. These two distributional processes can be differentiated because their intensities and time courses are different; image subtraction gives you the blood flow independent of previous dye accumulation. Redosing protocols are feasible since the dyes contemplated for use in these procedures have low toxicity and may be exploited to improve visualization of vasculature, space-occupying lesions, and biliary obstruction.

In cases of inflamed and sclerotic gall bladders, the surgeon's ability to differentiate between structures is reduced and the chance of unintended injury to the patient is increased. Problems are especially likely to arise with patients who present with relatively uncommon conditions, such as Mirizzi syndrome (a syndrome described in, for example, Chan et al., Surgeon, 1(5):273-8 (2003); Haritopoulos et al., Int. Surg. 87(2):65-8 (2002); and Pemberton and Wells, Postgrad Med J, 73:487-490 (1997)). The visualization of structures can be enhanced with administration of fluorescent dye prior to or during surgery: completely obstructed gall bladders will not accumulate the dye during cholecystectomy, but the extrahepatic, common hepatic, and common bile ducts will fluoresce. Further, the use of fluorescent dye facilitates endoscopic removal of common bile duct stones and the placement of endobiliary stents, since the duct will fluoresce and allow dye transfer is the duct is unobstructed.

Additionally, in cases of unequivocal biliary and gall bladder symptomatology, percutaneous imaging of the gall bladder may allow prompt exclusion of gall bladder obstruction without delayed laboratory tests or nuclear medicine imaging. Further, in patients with long standing right upper quadrant (“RUQ”) pain, one possible diagnosis is a dysfunctional gallbladder. Often, the real etiology of the pain cannot be confirmed even after subjecting the patient to multiple diagnostic tests. In such cases, laparoscopic cholecystectomy is often performed as a last resort. ICG may be imaged intraoperatively using NIRF and confirm or rule out the gall bladder as a source of symptoms.

The methods of the invention are also expected to be useful in improving the outcome of liver transplants. The methods permit improved preoperative assessment of donor livers for liver transplantation, and the intra- and post-operative evaluation of the donated liver in the recipient. As noted above, in pre-operative assessments, the dye is administered to the donor subcutaneously, and the fluorescence of the liver observed. Fluorescence throughout the liver indicates that the liver is fully perfused and more likely to be in good condition than a liver in which portions are not fluorescing. During implantation of the donor liver into the recipient, fluorescence of the liver subsequent to administration of dye into the recipient permits the practitioner to make blood flow assessments, and to find biliary leaks. In some cases, the intra-operative assessment is of a liver which has been resected in a living-related liver transplantation (see, e.g., Samstein and Emond, Ann. Rev. Med. 52: 147-160 (2001)). Post-implantation of the liver, fluorescence of the liver permits anastomotic blood flow assessments and finding of biliary leaks at the surface of living-related livers. Clearance of the dye from the circulation through the bile serves to verify that the liver is functioning. Typically, at the end of an operation, the surgical field, in this case the abdomen, is sutured shut, a process known as “closing” the surgical field. The assessments and verifications noted above are desirably performed before the surgical field is closed.

Administration of too much ICG to the donor may complicate post-transplantation evaluation. Hence, in some embodiments, SC or intramuscular (“IM”) administration of small doses, or continuous infusion of small doses, is preferable to systemic administration of a bolus of dye for determining adequate donor organ perfusion. The present invention therefore provides important new capabilities to the practitioner for the pre-operative assessment of donor livers, and the intra-, and post-operative assessment of the liver once it is transplanted into the recipient.

In another group of embodiments, the dye can be administered intravenously as a bolus to get a higher concentration. The practitioner then waits for the dye to be “washed out” of the normal parts of the liver by normal clearance mechanisms. Due to the abnormal vasculature in and permeability of the tumor, dyes tend to clear at a different rate from the tumor area than from normal liver tissue. As persons of skill are aware, a number of cancers of other organs (e.g., lung, colon or breast cancers), metastasize to the liver. In some instances, however, the cancer is a primary liver cancer—one originating in the liver itself.

The different types of tumor found in the liver are expected to have different characteristics, and to have different vascularization than normal liver, and to be more or less porous or permeable to the dye used for visualization depending on the tumor type (e.g., metastasized colon cancer, metastasized breast cancer, or primary liver cancer) compared to normal liver. All that is necessary for visualization of the tumor, however, is a difference between the tumor and normal liver tissue in vascularization and permeability. Thus, for example, if the blood vessels of the tumor are “leakier” to the dye than the blood vessels of normal liver, the tumor area will appear as a darker area than the surrounding, normal liver tissue. Conversely, if the vasculature of the tumor is “tighter” than that of normal liver, the dye will “wash out” of the normal liver faster than it washes out of the tumor and the tumor will appear more brightly fluorescent than does the normal liver. Whichever characteristic is true of the particular tumor in question, it is expected to be easily distinguished from the neighboring normal liver tissue. The time to wait for the dye to clear can be readily determined empirically by illuminating the liver with a light of the appropriate excitation frequency (discussed at greater length within), visualizing the emission fluorescence, and determining whether the fluorescence of most of the liver has diminished to a point where the tumor can be visualized relative to the normal liver tissue is possible.

### Instrumentation

Conveniently, the device used for visualization comprises both a laser and a camera. For convenience of reference, the discussion below refers to the exemplar dye ICG. Persons of skill will recognize that the other dyes mentioned herein as suitable for use in the inventive methods and procedures could be substituted for ICG, with the light source selected or adjusted to provide illumination optimized for the excitation frequency suitable for the particular dye chosen and the device for capturing the light emitted by the dye being selected or adjusted to be able to receive light of the appropriate frequency. For use with ICG, the laser conveniently consists of a laser diode providing a maximum of 3 W output at 806 nm. For other dyes, the laser diode is selected to provide a light with a wavelength at an excitation frequency appropriate for the dye selected.

The laser output is decollimated (i.e. optics are used to spread out the laser light from a tight beam) to provide even illumination over a field of view, for example, 7.6 cm by 7.6 cm at a working distance of 30 cm. The imaging system typically has a camera containing a charge-coupled device (“CCD”) or a complementary symmetry metal oxide semiconductor (“CMOS”) image sensor sensitive into the near infrared spectrum and, for use with ICG, is equipped with an 815 nm edge filter. In some embodiments, the laser or camera or both, are supported by an articulated arm connected to a wheeled base. This allows the imaging head to be moved into close proximity to the surgical table and for vertical movement of the head to attain an appropriate focal distance above the area of interest. The imaging head and extension arm that protrudes over the surgical field are typically covered with an optically transparent sterile drape. The laser can conveniently be activated by means of a computer command or by foot pedal. Laser/camera devices suitable for intra-operative imaging are commercially available. In some preferred embodiments, the laser/camera device is a SPY® Intra-operative Imaging System, a HELIOS® Imaging System, or a LUNA® Imaging System (all by Novadaq Technologies, Inc., Mississauga, Ontario, Canada).

In some embodiments, an instrument having an optical configuration similar to a fluorescence microscope may be used, in which a dichroic mirror is used to split the paths of the illumination (the excitation light). The excitation light reflects off the surface of the dichroic mirror into the objective, while the fluorescence emission passes through the dichroic mirror to the eyepiece or is converted into a signal to be presented on a screen. The instrument may further have an excitation filter or an emission filter, or both, to select the wavelengths appropriate for each function. Conveniently, the filters are interference filters, which block transmission of frequencies out of their bandpass.

For visualizing the area of interest, the considerations noted in the section above are taken into account, with the route and amount of dye varying according to the structure or function to be visualized. For example, to visualize smaller structures around the liver, or patency of a duct, the dye will usually be administered subcutaneously or intramuscularly, or by continuous intravenous infusion of small amounts. To visualize tumors in the liver, a systemic, larger bolus injection of dye may be administered, and the liver then illuminated with light of an appropriate excitation frequency to observe the differences in fluorescence between the tumor and the neighboring normal liver tissue. For the dye ICG, for example, an 806 nm excitation light causes the dye to fluoresce, emitting light at 830 nm. The emitted light can then be imaged directly or, preferably, is captured using an imaging system.

As noted, the capture system is typically a video camera containing a CCD or CMOS image sensor. The capture system feeds the image to a monitor so that the surgeon can visualize the fluorescence of the dye in the organ or area of interest in real time. Filters limit the light detected to a range appropriate for the selected fluorescence wavelengths. Optionally, the camera is also attached to a computer and the image is saved, which not only permits documentation of the fluorescence of the organ, but also can be used for training urologic surgeons, nurses, and other medical staff. Typically, the time required for positioning the device is 2 minutes, while the total time that the vessels are illuminated with laser light is 30 seconds.

The methods described herein are suitable for use in mammals. Examples of suitable mammals include, but are not limited to, humans, non-human primates, dogs, cats, sheep, cows, pigs, horses, mice, rats, rabbits, and guinea pigs. Use in primates, and particularly in humans, is preferred.

### Dyes for Imaging

As persons of skill are aware, fluorescent dyes have a particular excitation wavelength which causes the dye to fluoresce and emit light of a particular emission wavelength. Persons of skill will appreciate that a considerable literature is available in the art on the characteristics of different dyes, including their excitation wavelength and emission wavelength. This literature is well known, and will not be set forth in detail herein.

The dye is imaged by exciting it with a light that has an excitation wavelength appropriate for the particular dye used. Persons of skill are aware that a variety of dyes exist, and that each dye has an excitation wavelength and an emission wavelength. Some dyes, for example, fluoresce under ultraviolet (“UC”) illumination while others fluoresce under incandescent (visible light) illumination. The literature on the use of fluorescent dyes and probes in biological assays includes, for example, Dewey, T. G., Ed., Biophysical and Biochemical Aspects of Fluorescence Spectroscopy, Plenum Publishing (1991), Guilbault, G. G., Ed., Practical Fluorescence, Second Edition, Marcel Dekker (1990), Lakowicz, J. R., Ed., Topics in Fluorescence Spectroscopy: Techniques (Volume 1, 1991); Principles (Volume 2, 1991); Biochemical Applications (Volume 3, 1992); Probe Design and Chemical Sensing (Volume 4, 1994); Nonlinear and Two-Photon Induced Fluorescence (Volume 5, 1997); Protein Fluorescence (Volume 6, 2000); DNA Technology (Volume 7, 2003); Plenum Publishing, and Lakowicz, J. R., Principles of Fluorescence Spectroscopy, Second Edition, Plenum Publishing (1999) and W. T. Mason, ed., Fluorescent and Luminescent Probes for Biological Activity. A Practical Guide to Technology for Quantitative Real-Time Analysis, Academic Press (Second Ed., 1999).

Preferably, the dye selected is one that has low toxicity and has excitation and emission peaks within the “optical window” of tissue, where absorption due to endogenous chromophores is low. Preferred fluorescent dyes suitable for use in the methods of the invention are non-toxic dyes which fluoresce when exposed to radiant energy, e.g. light. Preferably, the dyes are near infrared fluorochromes, or “NIRF” that emit light in the near infra red spectrum. Near infrared light can penetrate tissue to a depth of several millimeters to a few centimeters. In some embodiments, the dye is a tricarbocyanine dye, and in particularly preferred embodiments, is ICG. In other embodiments the dye is selected from fluorescein isothiocyanate, rhodamine, phycoerythrin, phycocyanin, allophycocyanin, o-phthaldehyde, fluorescamine, Rose Bengal, trypan blue, and fluorogold. The dyes may be mixed or combined. In some embodiments, dye analogs may be used. A “dye analog” is a dye that has been chemically modified, but still retains its ability to fluoresce when exposed to radiant energy of an appropriate wavelength.

ICG, Fast Blue and Fluorogold have all been used in mammals with low evidence of toxicity and are preferred. As noted, ICG is particularly preferred both because it has low toxicity and because it has been approved by the Food and Drug Administration for several diagnostic purposes in humans. Further, its absorption (excitation) and emission peaks (805 and 835 nm, respectively) lie within the “optical window” of tissue. ICG is commercially available from, for example, Akom, Inc. (Buffalo Grove, Ill.), which sells it under the name IC-GREEN™. After intravenous injection, ICG is bound within 1 to 2 seconds, mainly to globulins (1-lipoproteins), and remains intravascular, with normal vascular permeability. ICG is not metabolized in the body and is excreted exclusively by the liver, with a plasma half-life of 3 to 4 minutes. It is not reabsorbed from the intestine and does not undergo enterohepatic recirculation. The recommended dose for ICG video angiography is 0.2 to 0.5 mg/kg.

For intraoperatively visualizing organs and structures of the hepatobiliary tree, the surgical field, or the portion of the surgical field in which imaging is desired, is illuminated with a light of the excitation wavelength or wavelengths suitable for the dye or dyes used. If desired, ambient light may be dimmed to facilitate visualization of the fluorescence. Where the excitation wavelength is outside of the visible range (where, for example, the excitation wavelength is in the ultraviolet or near infrared range), the light source may be designed to permit switching or “toggling” between the excitation wavelength and visible light. This permits the practitioner to note the position of fluorescent structures in relation to the rest of the surgical field and surrounding (but non-fluorescent) structures.

In some embodiments, an instrument having an optical configuration similar to a fluorescence microscope may be used, in which a dichroic mirror is used to split the paths of the illumination (the excitation light). The excitation light reflects off the surface of the dichroic mirror into the objective, while the fluorescence emission passes through the dichroic mirror to the eyepiece or is converted into a signal to be presented on a screen. The instrument may further have an excitation filter or an emission filter, or both, to select the wavelengths appropriate for each function. Conveniently, the filters are interference filters, which block transmission of frequencies out of their bandpass.

Dye administered systemically tends to result in high degrees of fluorescence of the vasculature, which can obscure light from the common bile duct, the common hepatic duct, the right hepatic duct, or other structures of interest. Thus, while systemic administration works well for visualization of the liver, where visualization of the common bile duct, the common hepatic duct, or the right hepatic duct is desired, the dye is preferably administered subcutaneously. Preferably, the dye is administered about 15 minutes before the practitioner expects to be exposing the liver, the bile duct, or other structures. The dye can, however, be administered during the surgery or up to about eight hours prior to the surgery, with about six hours before the surgery being preferred, about four hours before the surgery more preferred, about two hours still more preferred, and between 5 minutes to about one hour being still more preferred, with “about” in this context meaning the time can be one-half hour on either side of the designated time point.

The maximum daily dosage of ICG for adults is 2 mg/kg. There is no data available describing the signs, symptoms, or laboratory findings accompanying an overdose of ICG. The LD₅₀ after IV administration ranges between 60 and 80 mg/kg in mice, 50 and 70 mg/kg in rats, and 50 to 80 mg/kg in rabbits.

### EXAMPLES

### Example 1

Intraoperative video angiography is performed with a laser-fluorescence imaging device ((Novadaq Technologies, Inc., Mississauga, Ontario, Canada) consisting of a NIR laser light source and a NIRF-sensitive digital camcorder. For measurements, the unit is positioned 30 to 40 cm from the area of interest. ICG, dissolved in an appropriate carrier, such as saline solution, is then injected as a bolus. The NIR light emitted by the laser light source induces ICG fluorescence. The fluorescence is recorded by a digital video camera, with optical filtering to block ambient and laser light so that only ICG fluorescence is captured. Images can be observed on screen in real time (at approximately 25 to 30 images/sec (PAL or NTSC)). The images can be reviewed and stored on the digital video camera or transferred to a computer or to storage media.

### Example 2

In an initial clinical trial, fifteen (15) subjects are enrolled and assessed during the conduct of the trial. All study subjects receive standard of care assessments for their pre- and post-operative care. Candidates for this study meet all of the following inclusion criteria, and do not meet any of the listed exclusion criteria. The inclusion criteria are: subject (or legal representative) is willing and able to provide an informed consent; subject is willing and able to comply with the study procedures, a urine pregnancy test for women of reproductive age prior to surgery shows the woman is not pregnant, and the subject is scheduled for biliary surgery. The exclusion criteria are: subject has significant liver disease, cirrhosis or liver insufficiency with abnormal liver function tests, as total bilirubin>1.5×normal and/or SGOT>2×normal, subject has uremia, serum creatinine>2.5 mg/dl, subject has a previous history of adverse reaction or allergy to ICG, iodine, shellfish or iodine dyes, subjects in whom the use of x-ray dye or ICG is contraindicated including development of adverse events when previously or presently administered, subject has any medical condition, which in the judgment of the Investigator and/or designee makes the subject a poor candidate for the investigational procedure, subject is a pregnant or lactating female, or subject is participating in another drug, biologic and/or device protocol.

Once a subject has entered into the trial, the Investigator makes every reasonable effort to retain the subject in the trial. However, subjects may withdraw from the trial for any reason at any time. Investigators also may withdraw subjects from the trial (a) in order to protect subject safety and/or (b) if the subject is unwilling or unable to comply with required trial procedures.

At any time after ICG administration, if progressive impairment of liver and/or kidney function is diagnosed, the subject is withdrawn and referred for appropriate therapy. If a subject develops unacceptable toxicity, the subject is withdrawn or if at any time the subject is found to be ineligible for the protocol as designated in the inclusion and exclusion criteria.

The SPY™ Intra-operative Imaging System is utilized in an operating room setting and is covered with a sterile drape and moved into the surgical field. All surgical patients have an indwelling angiocath, or a central venous line, or both. Either access is acceptable for injection of ICG at the time of NIR imaging. Akorn, Inc. IC-Green™ is available as 6 each 25 mg vials and 10 ml ampoules of Aqueous Solvent. The usual adult dose of IC-Green™ is 5 mg of dye per ml of solution. Maximum daily dosage: 2 mg/kg ICG (Akorn, Inc.). The product is reconstituted with 10 ml of Aqueous Solvent provided, resulting in a final concentration of 2.5 mg/ml. Once reconstituted, the solution is used within ten hours.

### Example 3

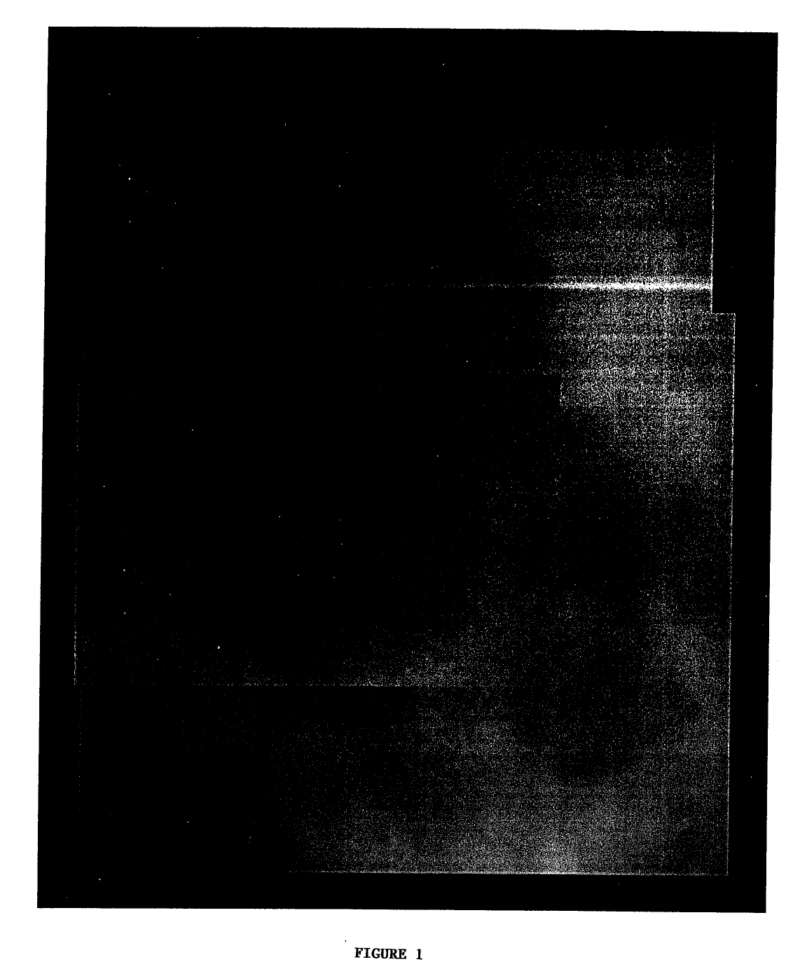

This Example reports the results of an investigation of liver visualization by fluorescent dye in an adult pig model. An adult pig (approximately 50-60 kilograms) was placed under appropriate anesthesia and its liver subjected to radiofrequency (RF) emissions from a RF needle electrode. The liver looked normal upon visual examination under normal light. ICG was administered intravenously as a bolus injection and the liver was visualized by fluorescent illumination using a SPY™ imaging system (Novadaq Technologies, Inc., Mississauga, Ontario, Canada). FIG. 1 is a composite of photographs taken of the light emitted from the liver under laser excitation light illumination. The lighter areas to the right and bottom sides of the photos indicate normal liver tissue. The darker areas in the middle shows areas of low perfusion, while the black area on the left side of the photos had no visible perfusion and was likely dead. Upon palpation, the light areas were flexible and normal, while the darker areas and the black area were relatively stiff.

### Example 4

This Example shows the liver of a male Sprague-Dawley rat visualized by laser excitation light delivered by fiber optics through a laparoscope. The brightly fluorescing structure is the liver; the bowel is to the right.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

What is claimed is:

1. A method of visualizing a hepatobiliary structure in a subject during a surgical operation, comprising:
(a) within eight hours prior to, or during, said surgical operation, administering by subcutaneous injection, intramuscular injection, or slow, continuous intravenous infusion, a dye which fluoresces at an emission wavelength when said dye is contacted with an excitation wavelength;
(b) exposing said hepatobiliary structure during said operation to a source of illumination comprising said excitation wavelength such that the fluorescent dye fluoresces; and
(c) detecting the fluorescence of said dye,
thereby visualizing said hepatobiliary structure during said surgical operation.

2. A method of claim 1, wherein said hepatobiliary structure is the bile duct, the common hepatic duct or the right hepatic duct.

3. A method of claim 1, wherein said hepatobiliary structure is the gall bladder or bile duct and the surgical operation is a cholecystectomy.

4. A method of claim 3, wherein said gall bladder or bile duct, or both, are inflamed.

5. A method of claim 1, wherein said hepatobiliary structure is the liver and the surgical operation is a resection of the liver for a living-related liver transplantation.

6. A method of claim 1, wherein said hepatobiliary structure is the liver and the surgical operation is transplantation of a donor liver.

7. A method of claim 1, wherein said hepatobiliary structure is the common bile duct and the surgical operation is removal of a stone from said bile duct.

8. A method of claim 1, wherein said surgical operation is by a laparoscopic instrument.

9. A method of claim 1, wherein said dye is administered one hour or less prior to said surgical operation.

10. A method of claim 1, wherein said dye is administered at the time of anesthesia induction.

11. A method of claim 1 wherein said dye is a near infrared dye.

12. A method of claim 11, wherein the near infrared dye is indocyanine green.

13. A method of determining, during a surgical operation to remove a patient's gall bladder, whether the patient's common bile duct is open following removal of the gall bladder, comprising:
(a) after the gall bladder has been removed, but during the surgical operation, administering systemically a bolus of a dye which fluoresces at an emission wavelength when said dye is contacted with an excitation wavelength;
(b) exposing said common bile duct to a source of illumination comprising said excitation wavelength such that the fluorescent dye fluoresces; and
(c) detecting the presence or absence of fluorescence of said dye throughout said common bile duct,
wherein presence of fluorescence of said dye throughout said common bile duct indicates that the common bile duct is open and wherein absence of fluorescence of said dye throughout said common bile duct indicates that the common bile duct is not open.

14. A method of claim 13, wherein said surgical operation is by a laparoscopic instrument.

15. A method of claim 13, wherein said dye is a near infrared dye.

16. A method of claim 15, wherein the near infrared dye is indocyanine green.

17. A method of assessing, during transplantation of a liver into an abdomen of a subject, blood flow in said transplanted liver, comprising:
(a) after said transplanted liver has had blood vessels of the subject connected to it, administering systemically before closing said abdomen a bolus of a dye which fluoresces at an emission wavelength when said dye is contacted with an excitation wavelength;
(b) exposing said liver to a source of illumination comprising said excitation wavelength such that the fluorescent dye fluoresces; and
(c) detecting the presence or absence of fluorescence of said dye throughout said liver,
wherein presence of fluorescence of said dye throughout said liver indicates that blood flow throughout the liver is satisfactory and wherein absence of fluorescence of said dye in some or all of said liver indicates that the blood flow is not satisfactory.

18. A method of claim 17, wherein said dye is a near infrared dye.

19. A method of claim 18, wherein the near infrared dye is indocyanine green.

20. A method of detecting the presence or absence of a lesion or tumor in a liver in a subject, comprising:
(a) administering to said subject a bolus of a dye which fluoresces at an emission wavelength when said dye is contacted with an excitation wavelength;
(b) providing an interval of time to permit said dye to partially wash out of said liver;
(c) exposing said liver to a source of illumination comprising said excitation wavelength such that any fluorescent dye in said liver fluoresces;
(d) detecting the presence or absence of fluorescence of said dye throughout said liver,
wherein presence of fluorescence of said dye evenly throughout said liver indicates the absence of an injury or tumor and differences in fluorescence between areas of said liver denote the presence of a lesion or tumor in said liver.

21. The method of claim 20, wherein an absence of fluorescence in a first area of said liver, which first area is bounded by a second area of said liver that is fluorescing, indicates the presence of a lesion in said first area.

22. The method of claim 20, wherein an abundance of fluorescence in a first area of said liver, which first area is bordered by a second, larger area comprising the majority of said liver that is fluorescing at a lower level than that of said first area, indicates the presence of a lesion in said first area.

23. The method of claim 20, wherein said dye is a near infrared dye.

24. The method of claim 23, wherein the near infrared dye is indocyanine green.
